(19) United States
(12) Patent Application Publication
(10) Pub. No.: US 2025/0295933 A1
(43) Pub. Date: Sep. 25, 2025

(54) COMBINED WEARABLE SUPPORT AND LOW LEVEL FAR-INFRARED RADIATION THERAPY DEVICE

(71) Applicant: Eduardo Marti, Sunrise, FL (US)

(72) Inventor: Eduardo Marti, Sunrise, FL (US)

(21) Appl. No.: 18/610,503

(22) Filed: Mar. 20, 2024

(51) Int. Cl. A61N 5/06; A61F 5/01

(52) U.S. Cl. CPC A61N 5/06; A61F 5/0123; A61N 2005/0645; A61N 2005/066

(57) ABSTRACT

A combined wearable support and radiation therapy device operable to provide support to a body part while also applying prolonged exposure to low intensity far-infrared radiation. The wearable support may be embodied as a support element for wearing on a body part of a wearer, such as a brace or orthotic, and a liner element or sleeve which is held between the support element and the body of a wearer. The wearable support's far-infrared treatment capabilities come from a bio-ceramic infusion which can be printed in a pattern on a surface of the liner element in such a way to provide for wide coverage of radiation and air flow. In this regard, the combined wearable support and radiation therapy device provides the far-infrared radiation treatment while at the same time continuing to providing structural and range of motion support to the wearer.

### BACKGROUND OF THE INVENTION

### Field of the Invention

This invention relates generally to a wearable supports and orthotic devices and, more particularly, to a wearable support or orthotic device having a bio ceramic infused surface uses body heat to provide a prolonged, therapeutic exposure of low level far-infrared radiation to tissues inside the wearable support or orthotic device.

### Description of the Prior Art

An injury to, or a surgical procedure on, a limb and/or joint is typically followed by a period of rehabilitation in order to aid recovery. Post-operative rehabilitation of the joint has been shown to reduce the likelihood of outcomes like scar tissue build up, reduced mobility and muscle atrophy. Joint rehabilitation further promotes faster healing, reduction of the amount of pain experienced, and improvement in the range of motion (or “ROM”) of the affected joint.

An important aspect of rehabilitation is being able to use the joint while providing it with the correct amount of support and protection to prevent re-injury at the time when full strength has yet to be realized. For example, in the case of the knee, this support and protection is usually provided in the form of patella support as well as ROM control.

There are various methods that can be used to improve post injury and/or post operative recovery. Beyond use of the joint and standard rehabilitation activities, one method found to be effective in aiding a recovery process is the application of far-infrared radiation (or “FIR”) treatment. FIR therapy causes the production of Nitric oxide when the radiant energy enters the body, stimulating the capillaries to expand and in doing so increases blood flow which improves cellular regeneration and increases oxygen supply, all of which are important to localized healing as well as systemic healing. FIR also can penetrate deeper into the skin which helps activate fibroblasts to release more collagen, which is a very important part of wound healing and soft tissue repair. This release of collagen that makes FIR excellent for healing burns, scar tissue and skin problems such as acne and skin breakouts. There are also anti-oxidative properties which promote anti-aging and lessening/softening of wrinkles due to this enhanced production of collagen.

In the context of the recovery process, FIR can treatment assist by providing enhanced blood circulation and improved oxygenation in the affected area as well as reduced swelling. In addition, a reduction in swelling can be experienced as well as local detoxification due to the increase in circulation and improved oxygenation. Benefits are also seen in the improvement of pain management as well as overall systemic healing. FIR treatment can be provided short term or long term and with various levels of intensity to achieve these types of benefits.

Applying FIR for treatment is often done through a mechanism that utilizes an external energy source. For example, LED lights have been used to provide FIR treatments. A concern with such applications, however, is that there are limitations on the frequency and/or duration of the same.

Tourmaline is a bio-ceramic material which also can be used to generate FIR. Tourmaline has the unique ability to absorb body heat, convert it to far infrared radiation and, radiate the far infrared radiation back out. In comparison to FIR treatment generated through a medium that utilizes an external energy source, such as LED FIR treatment, FIR treatment generated by converting body heat through the use of tourmaline is considered a low intensity application of FIR. Prolonged exposure to such a level of FIR is therefore safe and considered beneficial as heat or radiation levels will not be high enough to burn or damage skin tissue but rather provide the desired effects of promoting improved recovery through improved blood circulation and reduced swelling in the affected area.

Thus, there remains a need for a device which can provide an injured and/or operated on a body part with both prolonged, low level FIR treatment and joint protection and support during the recovery and rehabilitation process. It would be additionally desirable for such a device to remain breathable despite the application of FIR treatment. It would be further desirable if such a device was able to more securely remain in place on a body part.

### SUMMARY OF THE INVENTION

The present disclosure concerns wearable supports such as orthotic devices which have an infusion of bio-ceramic material into silicone paint that is integral therewith for the purpose of assisting with rehab in post operative and post injury scenarios. Assemblies provided for herein may combine the structural and ROM support provided by the wearable support with far-infrared radiation treatment that is supplied by the bio-ceramic infused silicone paint to assist with the recovery process.

In this regard, the present disclosure provides for a combined wearable support and radiation therapy device for use on a wearer, comprising: a support element adapted to be positioned on and removably secured to a part of the body of a wearer; a liner element, wherein when the support element is positioned on the body of the wearer, the liner element is adapted to be positioned between the support element and the body of the wearer with at least a portion of the liner element contacting the wearer; and a radiation generating treatment integral with the liner element, wherein when the liner element is positioned between the support element and the body of the wearer, the radiation generating treatment is operative to generate radiation from heat from the body of the wearer and cause radiation to be directed towards the body of the wearer.

It is an object of the present disclosure to provide a combined wearable support and radiation therapy device which provides an injured and/or operated on a body part such as a joint with both prolonged, low level FIR treatment and joint protection and support during the recovery and rehabilitation process.

It is an additional object of the present disclosure to provide a combined wearable support and radiation therapy device which remains breathable despite the application of FIR treatment.

It is yet another object of the present disclosure to provide a combined wearable support and radiation therapy device which is able to more securely remain in place on a body part such as a joint.

### BRIEF DESCRIPTION OF THE DRAWINGS

FIG. 1 is a front elevational view of a combined wearable support and radiation therapy device built in accordance with a knee brace embodiment the present invention, shown in place on a wearer.

FIG. 2 is a front elevational view of a combined wearable support and radiation therapy device built in accordance with a knee brace embodiment the present invention.

FIG. 3 is a side elevational view of a combined wearable support and radiation therapy device built in accordance with a knee brace embodiment the present invention.

FIG. 4 is a rear perspective view of a combined wearable support and radiation therapy device built in accordance with a knee brace embodiment the present invention.

FIG. 5 is a top plan view of a sleeve element of a combined wearable support and radiation therapy device built in accordance with a knee brace embodiment the present invention, shown with the top spread open to expose the liner element.

FIG. 5A is a partial plan view of the interior surface of a sleeve element of a combined wearable support and radiation therapy device built in accordance with a knee brace embodiment the present invention.

FIG. 6 is a top plan view of an exemplary liner element of a combined wearable support and radiation therapy device built in accordance with the present invention, shown with the liner element exposed.

FIG. 7 is a rear perspective view of a combined wearable support and radiation therapy device built in accordance with an LSO embodiment the present invention, shown in a closed configuration in place on a wearer.

FIG. 8 is a front perspective view of a combined wearable support and radiation therapy device built in accordance with an LSO embodiment the present invention, shown in a closed configuration in place on a wearer.

FIG. 9 is a front elevational view of a combined wearable support and radiation therapy device built in accordance with an LSO embodiment the present invention, shown in an open configuration.

FIG. 10 is a front elevational view of a combined wearable support and radiation therapy device built in accordance with an LSO embodiment the present invention, shown in an open configuration with its removed liner elements.

FIG. 11 is a rear elevational view of a back brace element of a combined wearable support and radiation therapy device built in accordance with an LSO embodiment the present invention, shown in an open configuration.

FIG. 12 is a rear elevational view of a center liner element of a combined wearable support and radiation therapy device built in accordance with an LSO embodiment the present invention.

FIG. 13 is a front perspective view of a center liner element of a combined wearable support and radiation therapy device built in accordance with an LSO embodiment the present invention, shown with its removed center support body.

FIG. 14 is a rear elevational view of side liner elements of a combined wearable support and radiation therapy device built in accordance with an LSO embodiment the present invention.

FIG. 15 is a rear perspective view of a combined wearable support and radiation therapy device built in accordance with a lumbar corset embodiment the present invention, shown in a closed configuration in place on a wearer.

FIG. 16 is a front perspective view of a combined wearable support and radiation therapy device built in accordance with a lumbar corset embodiment the present invention, shown in a closed configuration in place on a wearer.

FIG. 17 is a front elevational view of a combined wearable support and radiation therapy device built in accordance with a lumbar corset embodiment the present invention, shown in an open configuration.

FIG. 18 is a rear elevational view of a combined wearable support and radiation therapy device built in accordance with a lumbar corset embodiment the present invention, shown in an open configuration.

FIG. 19 is a front elevational view of a combined wearable support and radiation therapy device built in accordance with a lumbar corset embodiment the present invention, shown in an open configuration with its removed liner element.

FIG. 20 is a rear perspective view of a liner element of a combined wearable support and radiation therapy device built in accordance with a lumbar corset embodiment the present invention.

### DETAILED DESCRIPTION OF THE INVENTION

Described herein is a wearable support (such as an orthotic device) with far-infrared treatment capabilities, operable to provide support to a body part while also applying prolonged exposure to low intensity far-infrared radiation. Such an application can assist in promoting improved blood circulation, oxygenation, detoxification as well as reduction in swelling of the affected area which can lead to local and systemic healing.

The wearable support's far-infrared treatment capabilities come from a bio-ceramic coated or impregnated material integral therewith. The bio-ceramic coated material or impregnated material may be defined as a silicone paint which has been infused with a bio-ceramic material. This bio-ceramic infusion can be placed on a surface of the wearable support, thereby providing the far-infrared treatment capabilities without compromising the ability of the wearable support to provide to the structure support function of the wearable support.

It is contemplated that such a bio-ceramic infusion can be used on a multitude of wearable supports. Illustrated herein is an example of the bio-ceramic infusion being advantageously used in conjunction with a knee brace assembly, a lumbar sacral orthosis, and a lumbar corset assembly. Incorporation of the bio-ceramic infusion into such assemblies allows for a low level prolonged exposure to far-infrared radiation (or “FIR”) which promotes improved healing of soft tissue after injury or in post op scenarios.

The bio-ceramic infusion may consist of a tourmaline powder mixed at a 20% ratio to a silicone paint. This ratio allows for the maximum effect of the tourmaline powder to apply the FIR treatment, while still maintaining sufficient flexibility to be used on wearable devices and allow for bending and free movement without rigid causing an undesirable level of rigidity or even breaking off as the wearable device moves due to the wearer.

A bio-ceramic infusion formed from silicone paint mixture that is 20% tourmaline powder may be used to cover an aspect of a wearable device. When a wearable device having the bio-ceramic infusion is worn, FIR is generated due to the integrated tourmaline powder from the body heat of the wearer. Thus, the distributed tourmaline powder in the infusion is used to cause the application of FIR to the patient when wearing the device.

Another important aspect of the bio-ceramic infusion is the manner in which it may be applied to a wearable device. The use of a bio-ceramic infusion on the surface of a wearable could cause the wearable to become too hot and otherwise uncomfortable. In order to provide comfort and allow for heat dispersion and allow for sufficient airflow to provide some cooling while the wearable is worn, while still providing evenly dispersed and complete coverage of the FIR, the bio-ceramic infusion may be applied in a pattern. This pattern may be defined by a repeating pattern of section of bio-ceramic infusion coated surface positioned next to a surface where there is no bio-ceramic infusion. Such a design allows for heat to escape through the spaces between the sections where there is bio-ceramic infusion, and for relatively cool air to flow in, thereby preventing the bio ceramic silicone infusion from causing too much heat to accumulate.

A bio-ceramic infusion disposed on the surface of the wearable may also offer a secondary benefit of providing enhanced gripping for the wearable device to help prevent it from sliding around when fitted to the user. The use of a pattern may enhance this as it allows moisture to escape and also creates a textured gripping surface. Thus, the structure which operates to apply FIR treatment also allows for a device may be better fitted and equipped to provide its mechanical benefits. As such, a device built in accordance with this disclosure not only can offers the substantial benefits of FIR treatment to the recovering joint, it can provide an added measure of security to prevent against reinjury.

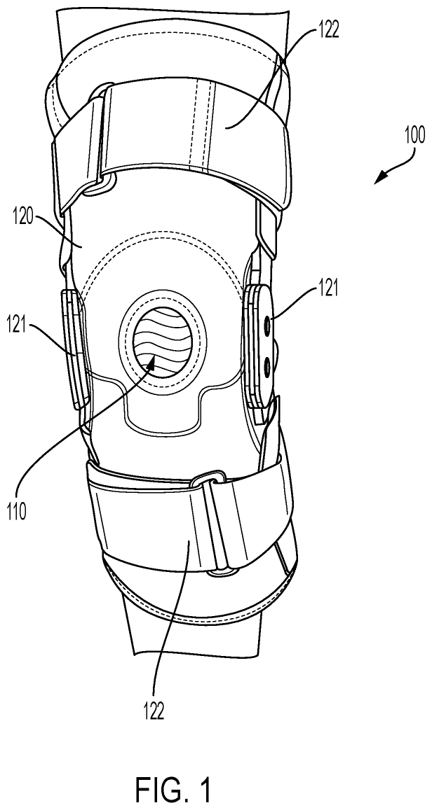

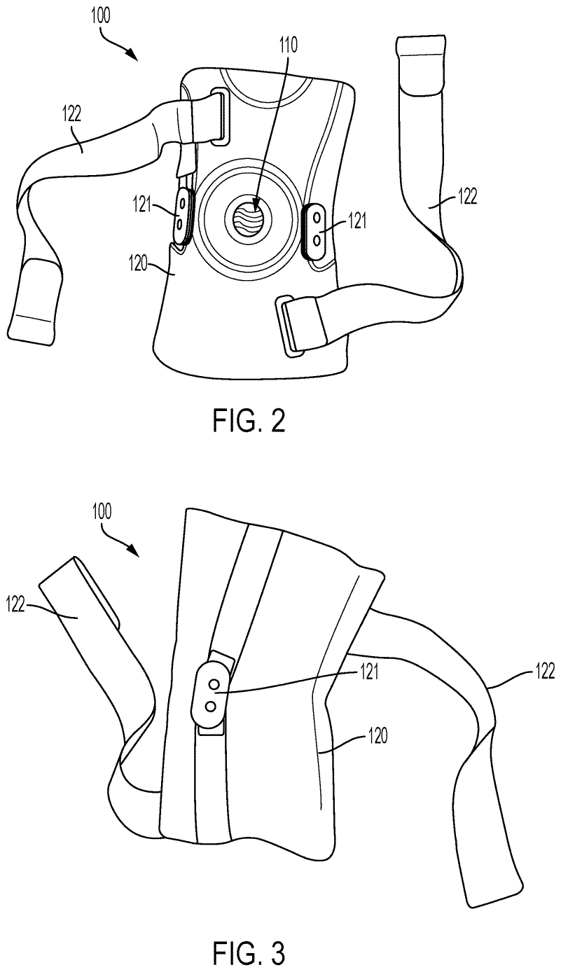

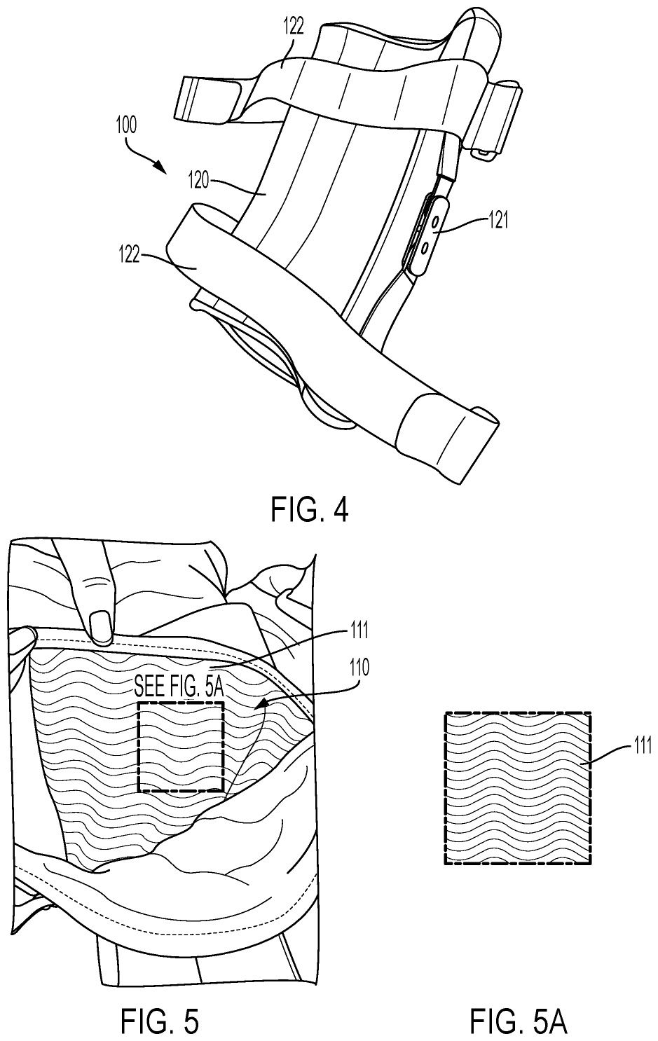

Referring now to the drawings, and, in particular, FIGS. 1, 2, 3, 4, 5, 5a, and 6, a knee brace embodiment of a combined wearable support and radiation therapy device 100 may include a liner element 110 and a knee brace element 120. The liner element 110 may be defined as a thin planar body which is constructed of a flexible fabric. In this regard, the liner element 110 is adapted to be positioned against the skin of a wearer and contour to the surface of the skin. In the illustrated embodiment, the liner element 110 is sewn to the inside or otherwise permanently attached to the inside of the knee brace element 120, thereby operating as an inner surface of the knee brace element 120. This arrangement allows for the liner element 110 to be placed against the skin of a wearer with the knee brace element 120 surrounding it and securing it against the skin when the combined wearable support and radiation therapy device 100 is worn.

In some embodiments, the liner element 110 may be removably attachable to the knee brace element 120 through the use of corresponding hook and loop fastener surfaces or other securing structures.

The knee brace element 120 may be sized and shaped to be placed over a knee of a wearer, as illustrated in in FIG. 1, and thereby adapted to be placed over the knee of the wearer. The knee brace element 120 may further be contoured on either side by ROM controlling struts, such as hinges 121. The hinges 121 may be held in place through conventional means, such as by securing pockets on the knee brace element 120. The knee brace element 120 may be operative to be secured in place on a wearer also through conventional means, such as adjustable straps 122 located above and below the knee. In this regard, the knee brace element 120 is adapted to be removably secured to the knee. In addition to allowing for the knee brace element 120 to be selectively tightened and loosened, the adjustable straps 122 may also operate to reinforce the hinges 121.

The liner element 110 is the aspect of the combined wearable support and radiation therapy device 100 on which a bio ceramic infusion 111 may be applied. This arrangement allows the bio ceramic infusion 111 to be as close to the skin as possible. In embodiments wherein the bio ceramic infusion 111 is disposed on the inside (i.e., skin side) of the liner element 110, it can be in direct contact with the wearers skin. In alternate embodiments, the bio ceramic infusion 111 may be on either or both sides (skin side, brace side) of the liner element 110. In embodiments wherein the bio ceramic infusion 111 in on both sides, it is contemplated that the pattern may be offset such that the open sections on one side are horizontally aligned with the coated sections on the other side or aligned with the open sections on one side are horizontally aligned with the open sections on the other side.

In this embodiment, the liner element 110 operates as an inner surface that includes the bio ceramic infusion 111 which is intended to be in contact with a wearer's skin. As such, when the liner element 110 is opened up, turned inside out, or folded back when in place on a wearer, the bio ceramic infusion 111 is exposed. As noted above, the bio ceramic infusion 111 may be defined by a mixture formed from 20% Tourmaline powder and the rest silicone paint and operates the generate the FIR. When disposed on the portion of the liner element 110 operating as an inner surface to the combined wearable support and radiation therapy device 100, this bio ceramic infusion 111 is situated to provide the FIR treatment effect on the wearer and while operating as the first level of contact with the wearer's skin.

The bio ceramic infusion 111 may be printed on the liner element 110 in pattern defined by printed bands of bio ceramic infusion 111 forming repeating strips that are 5.5 mm wide and are followed by a 5.5 mm wide space having no bio ceramic infusion 111 or other coating on their surface. These strips may be shaped in a wave pattern for not just aesthetic purposes (although the particular pattern selected, relative to some other pattern, may be for aesthetics), but also to better allow for an even distribution of FIR to the affected area. The even spacing and distribution of the bio ceramic infusion 111 additionally ensures improved air flow to the skin of the user as well as allows for excess heat to escape. The bio-ceramic bio ceramic infusion 111 also assists the brace from slipping on the user and keeps it in the desired position. These factors combine to provide for an effective treatment as well as a comfortable wearing product that can be used for extended periods of time.

As the knee brace embodiment concerns a wearable for a knee joint, it would be to provide knee support to the user, as well as ROM control through the use of the padded knee brace element 120 that works in conjunction with ROM hinges 121. It is contemplated, however, that the specific nature of the mechanical and ROM support would depend on the type of brace to which the bio-ceramic infusion is used with. In the knee brace embodiment, the knee brace element 120 and liner element 110 operate together to form a device which can apply the FIR treatment while also providing support the patella as well as mechanically defining the ROM of a patient. It is contemplated that the principle of a wearable support with a separate liner or integrated interior surface containing a bio ceramic infusion can be applied to other orthotic devices and allow them to provide low level prolonged FIR treatment to a portion of the body in addition to the primary functions of the given orthotic device. It is appreciated, however, that the use of a separate liner may allow for easier cleaning, disinfecting, and/or deodorizing of the skin contacting (and sweat generating) aspect of a combined wearable support and radiation therapy device built in accordance with the present invention.

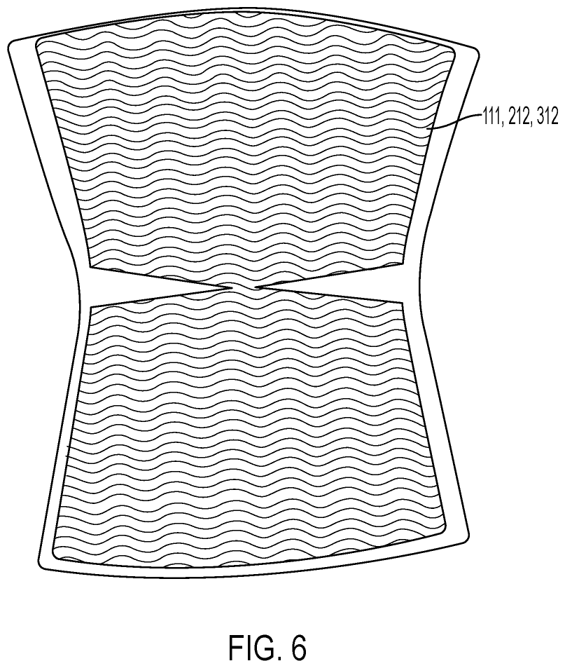

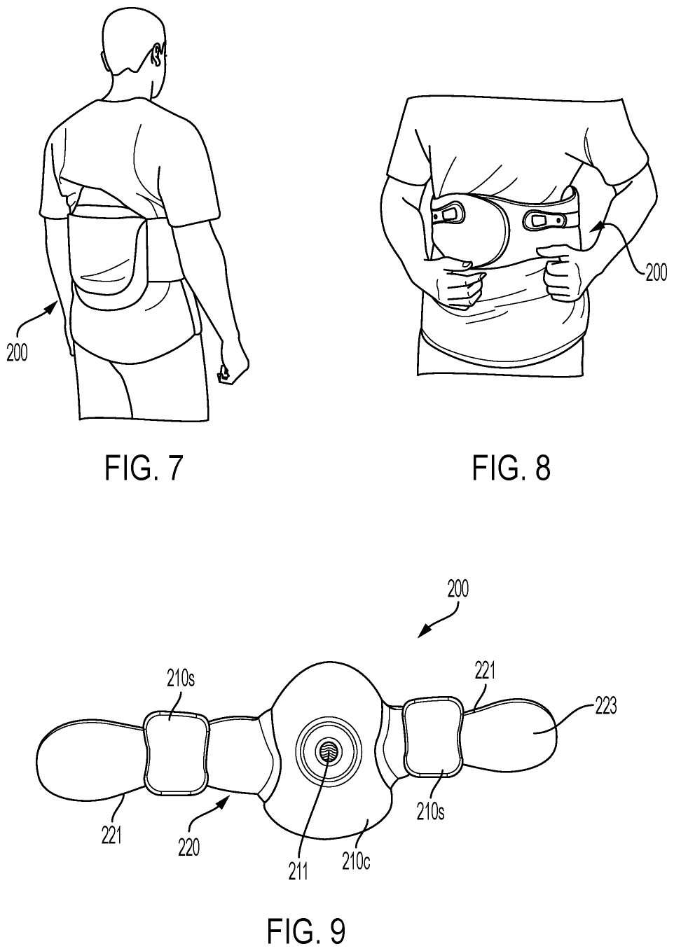

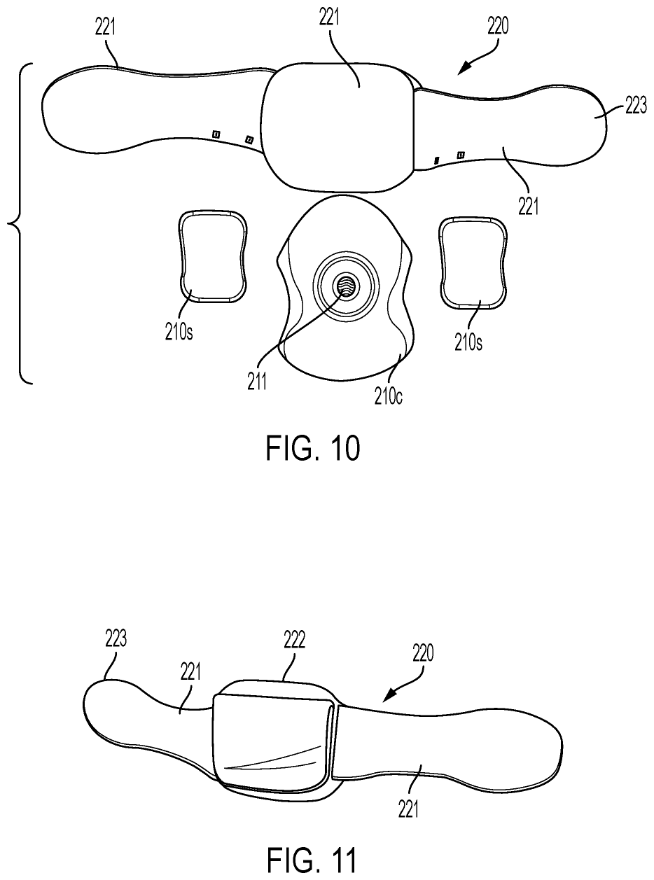

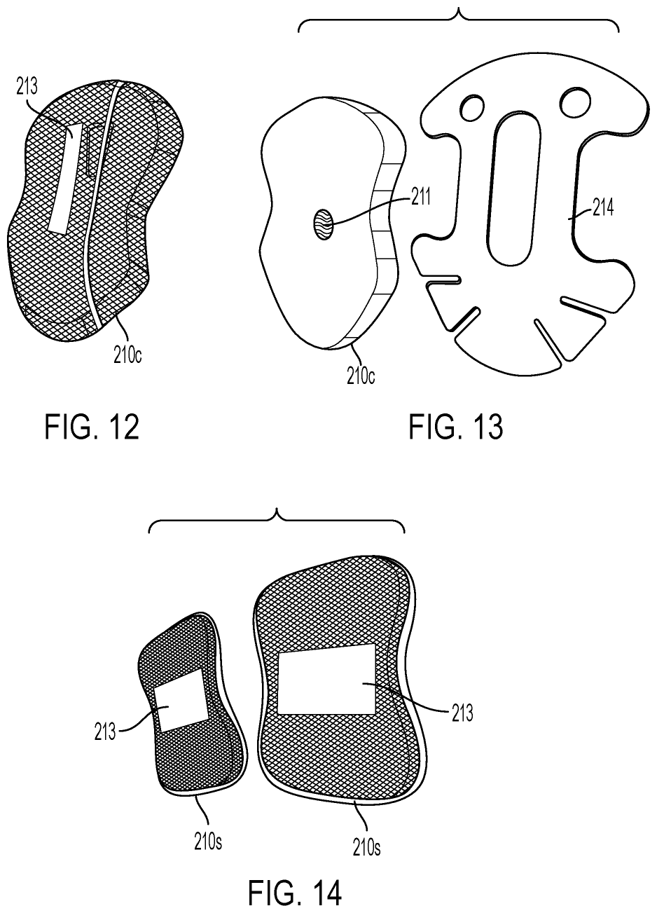

Referring now to FIGS. 6, 7, 8, 9, 10, 11, 12, 13 and 14, a combined wearable support and radiation therapy device 200 may alternatively be embodied as a lumbar sacral orthosis. In such an LSO embodiment, the combined wearable support and radiation therapy device 200 may include a plurality of liner elements 210 and a back brace element 220. The liner elements 210 may each be defined as a rigid, thin member which includes a bio ceramic infusion 212 integral therewith. In this regard, the liner elements 210 are adapted to be positioned against the body of a wearer.

The back brace element 220 may be structured as a conventional lumbar sacral orthosis or LSO style back brace, and thereby configured to be wrapped and secured in place around the torso of a wearer with one or more rigid support elements being held in place against the body of the wearer. In this regard, the back brace element 220 in accordance with the present disclosure includes a pair of opposing attachment arms 221 and a rigid lumbar support 222. The attachment arms 221 may each include corresponding securing areas 223, which each may be defined as an area of hook and loop fastener surface. The back brace element 220 may be operative to be removably secured in place on a wearer by placing the lumbar support 222 against the wearer's lumbar area of their back, extending the attachment arms 221 around to the front of the wearers body, and engaging the respective securing areas 223 on the attachment arms 221 to attach the arms together around the wearer's torso.

The combined wearable support and radiation therapy device 200 in illustrated embodiment includes three liner elements 210, each of which can be removably secured to the back brace element 220. A center liner element 210c is sized and contoured to serve as a back panel. Two side liner elements 210s are additionally included, each having a substantially rectangular profile. The side line elements 210s are each suitable to attach to one of the attachment arms 221, allowing them to be positioned against the side of a wearer when the combined wearable support and radiation therapy device 200 is worn by the wearer.

Each of the liner elements 210 includes a liner sheet 211 on which the bio ceramic infusion 212 may be applied. In the illustrated embodiment, the liner sheet 211 in each liner element 210 is defined by a thin sheet having printed thereon bands of a bio ceramic infusion 212. As with the knee brace embodiment, the bands of bio ceramic infusion 212 may be defined by repeating strips that are 5.5 mm wide and are followed by a 5.5 mm wide space having no bio ceramic infusion or other coating on their surface.

The liner elements 210 of the LSO embodiment may additionally include a mesh cover which encloses the liner sheet 211, a rigid support body which gives the liner element 210 its rigidity, and an attachment surface 213 for releasably attaching the liner elements 210 to the back brace element 220. The center liner element 210c may employ a center support body 214 as its rigid support body. The side liner elements 210s may employ a rigid support body having the same overall profile as the side liner elements 210s themselves. Embodiments of the liner elements 210 may employ the hook portion of a hook and loop fastener as the attachment surface 213, with the back brace element 220 having disposed on it some fabric material suitable to serve as the loop portion of a hook and loop fastener.

It is contemplated that the mesh cover may function to provide padding in the liner elements 210.

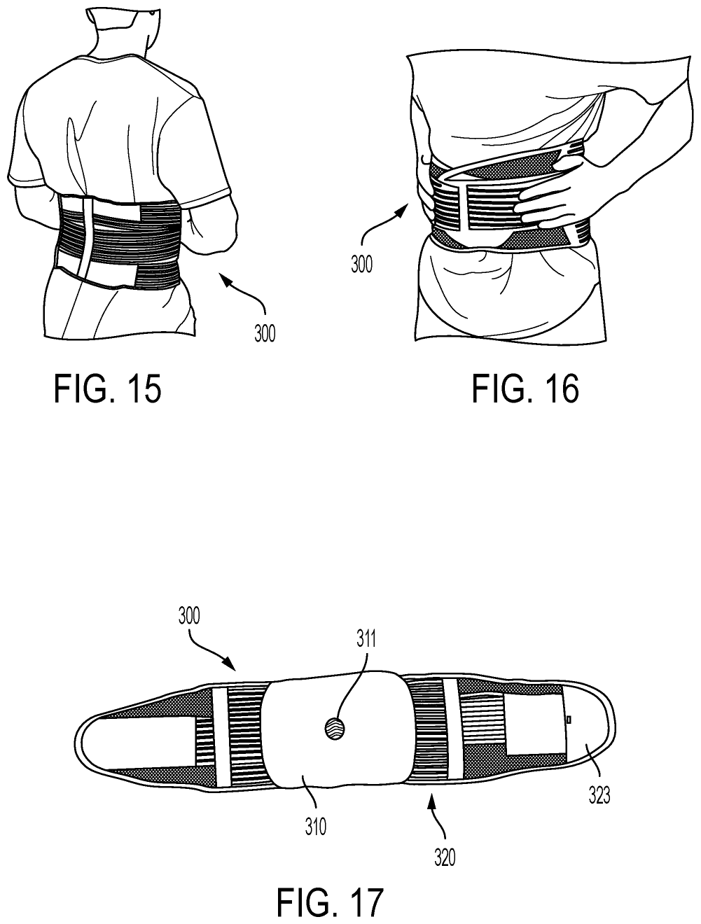

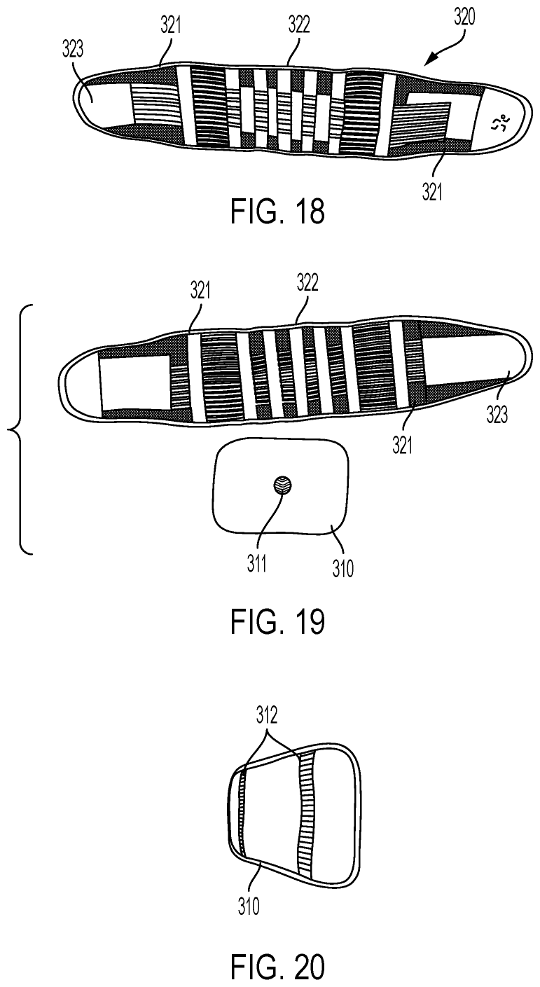

Referring now to FIGS. 6, 15, 16, 17, 18, 19 and 20, a combined wearable support and radiation therapy device 300 may alternatively be embodied as a lumbar corset. In such a lumbar corset embodiment, the combined wearable support and radiation therapy device 300 may include a liner element 310 and a corset element 320. The liner element 310 may be defined as a thin flexible liner sheet 311 which includes a bio ceramic infusion 312 integral therewith. In this regard, the liner element 310 is adapted to be positioned against the body of a wearer.

The corset element 320 may be structured as a conventional lumbar corset, and thereby be constructed of a substantially flexible material and structured to be wrapped and secured in place around the torso of a wearer. In this regard, the corset element 320 in accordance with the present disclosure includes a pair of opposing attachment arms 321 extending outward from a center portion 322. The attachment arms 321 may each include corresponding securing areas 323, which each may be defined as an area of hook and loop fastener surface. The corset element 320 may be operative to be removably secured in place on a wearer by placing the center portion 322 against the wearer's lumbar area of their back, extending the attachment arms 321 around to the front of the wearers body, and engaging the respective securing areas 323 on the attachment arms 321 to attach the arms together around the wearer's torso.

The liner element 310 can be removably secured to the center portion 322 and is sized to cover a significant portion of the lower back of a wearer. The liner elements 310 includes a liner sheet 311 on which the bio ceramic infusion 312 may be applied. In the illustrated embodiment, the liner sheet 311 is defined by a thin sheet having printed thereon bands of a bio ceramic infusion 312. As with the knee brace embodiment and the LSO embodiment, the bands of bio ceramic infusion 312 may be defined by repeating strips that are 5.5 mm wide and are followed by a 5.5 mm wide space having no bio ceramic infusion or other coating on their surface.

The liner element 310 may additionally include a mesh cover which encloses the liner sheet 311 and an attachment surface 312 for releasably attaching the liner element 310 to the center portion 322. Embodiments of the liner element 310 may employ the hook portion of a hook and loop fastener as the attachment surface 312, with the center portion 322 having disposed on it some fabric material suitable to serve as the loop portion of a hook and loop fastener.

It is contemplated that the mesh cover may function to provide padding in the liner element 310.

A key benefit of a combined wearable support and radiation therapy device in accordance with an embodiment of the present disclosure is that it can provide the benefits of the low level prolonged FIR treatment, while also providing the other desired functions of the wearable support. Taking the knee brace device as an example, the FIR treatment can be delivered and simultaneous the device also utilizes the padded knee brace element to provide support to the patella and surrounding soft tissue. This knee support with the aid of the ROM hinges also allows for ROM control, preventing over extension of the knee and keeping knee tracking in alignment. In this regard, FIR treatment may be applied simultaneously as the other functions of the required orthotic device without interfering in their functionality, allowing the user the benefits of combined treatment and accelerated healing while still providing joint and soft tissue support and protection.

The instant invention has been shown and described herein in what is considered to be the most practical and preferred embodiment. It is recognized, however, that departures may be made therefrom within the scope of the invention and that obvious modifications will occur to a person skilled in the art.

What is claimed is:

1. A combined wearable support and radiation therapy device for use on a wearer, comprising:
a support element adapted to be positioned on and removably secured to a part of the body of a wearer;
a liner element, wherein when the support element is positioned on the body of the wearer, the liner element is adapted to be positioned between the support element and the body of the wearer with at least a portion of the liner element contacting the wearer; and
a radiation generating treatment integral with the liner element, wherein when the liner element is positioned between the support element and the body of the wearer, the radiation generating treatment is operative to generate radiation from heat from the body of the wearer and cause radiation to be directed towards the body of the wearer.

2. The combined wearable support and radiation therapy device of claim 1, wherein the radiation generating treatment includes a bio-ceramic material.

3. The combined wearable support and radiation therapy device of claim 2, wherein the bio-ceramic material is defined by Tourmaline powder.

4. The combined wearable support and radiation therapy device of claim 2, wherein the radiation generating treatment includes a silicone paint.

5. The combined wearable support and radiation therapy device of claim 4, wherein the radiation generating treatment is formed from a mixture of the bio-ceramic material silicone paint that is no more than twenty percent bio-ceramic material.

6. The combined wearable support and radiation therapy device of claim 2, wherein the radiation generated from heat includes far-infrared radiation.

7. The combined wearable support and radiation therapy device of claim 1, wherein:
the liner element includes a treatment surface; and
the radiation generating treatment is disposed on the treatment surface.

8. The combined wearable support and radiation therapy device of claim 7, wherein the liner element is adapted to be positioned between the support element and the body of the wearer with at least a portion of the treatment surface contacting the wearer.

9. The combined wearable support and radiation therapy device of claim 1, wherein the radiation generating treatment is integral with the liner element in a pattern in which segments of the liner element containing the radiation generating treatment are interspersed with segments of the liner element containing none of the radiation generating treatment.

10. A combined wearable support and radiation therapy device for use on a wearer, comprising:
a support element adapted to be positioned on and removably secured to a part of the body of a wearer;
a liner element having a treatment surface, wherein when the support element is positioned on the body of the wearer, the liner element is adapted to be positioned between the support element and the body of the wearer with at least a portion of the treatment surface adjacent to the wearer; and
a radiation generating treatment integral with the treatment surface, wherein when the liner element is positioned between the support element and the body of the wearer, the radiation generating treatment is operative to generate radiation from heat from the body of the wearer and cause radiation to be directed towards the body of the wearer.

11. The combined wearable support and radiation therapy device of claim 10, wherein the radiation generating treatment includes a bio-ceramic material.

12. The combined wearable support and radiation therapy device of claim 11, wherein the bio-ceramic material is defined by Tourmaline powder.

13. The combined wearable support and radiation therapy device of claim 11, wherein the radiation generating treatment includes a silicone paint.

14. The combined wearable support and radiation therapy device of claim 13, wherein the radiation generating treatment is formed from a mixture of the bio-ceramic material silicone paint that is no more than twenty percent bio-ceramic material.

15. The combined wearable support and radiation therapy device of claim 11, wherein the radiation generated from heat includes far-infrared radiation.

16. The combined wearable support and radiation therapy device of claim 10, wherein the liner element is adapted to be positioned between the support element and the body of the wearer with at least a portion of the treatment surface contacting the wearer.

17. The combined wearable support and radiation therapy device of claim 10, wherein the radiation generating treatment is disposed on the treatment surface of the liner element is a pattern in which segments of the treatment surface containing the radiation generating treatment are interspersed with segments of the treatment surface containing none of the radiation generating treatment.

18. The combined wearable support and radiation therapy device of claim 10, wherein the radiation generating treatment is disposed on the treatment surface of the liner element is a repeating pattern in which elongated strips the radiation generating treatment are interspersed with segments of the treatment surface containing none of the radiation generating treatment.

19. The combined wearable support and radiation therapy device of claim 18, wherein the elongated strips the radiation generating treatment are defined by waves.

20. A combined wearable support and radiation therapy device for use on a wearer, comprising:
a knee brace element adapted to be positioned on and removably secured around a knee of a wearer;
a sleeve element having an inner surface and an outer surface, wherein when the knee brace element is positioned on the body of the wearer, the sleeve element is adapted to be positioned around the knee of the wearer between the knee brace element and the knee of the wearer with at least a portion of the inner surface contacting the wearer and with at least a portion of the outer surface contacting the knee brace element; and
a radiation generating treatment integral with the sleeve element, wherein when the sleeve element is positioned around the knee of the wearer, the radiation generating treatment is operative to generate radiation from heat from the wearer and cause radiation to be directed towards the wearer.
